(19) United States
(12) Patent Application Publication
(10) Pub. No.: US 2012/0245230 A1
(43) Pub. Date: Sep. 27, 2012

(54) METHOD AND COMPOSITION FOR PREPARING STABLE LIQUID FORMULATIONS OF PARACETAMOL

(75) Inventors: Dina Stela Velez Ferreira, Lisboa (PT); João Pedro Silva Serra, Cascais (PT); Nuno Miguel Araüjo Quintal, Odivelas (PT)

(73) Assignee: TECNIMEDE-SOCIEDADE TECNICO- MEDICINAL, S.A., Sintra (PT)

(21) Appl. No.: 13/514,772

(22) PCT Filed: Dec. 10, 2009
(86) PCT No.: PCT/PT2009/000069

§ 371 Date: Jun. 8, 2012

(51) Int. Cl. A61K 31/167; B65B 31/00; A61P 29/00

(52) U.S. Cl. 514/629; 53/403

(57) ABSTRACT

The present invention relates to stable liquid formulations of paracetamol for pharmaceutical use and to a method of preparation of stable paracetamol solutions.

### TECHNICAL FIELD

The present invention refers to stable liquid formulations of paracetamol for pharmaceutical use and to a method of preparation of stable paracetamol solutions.

### BACKGROUND

It is common knowledge that there exist some active ingredients which present stability problems in solution. Some of these problems are due to the fact that the active ingredients easily oxidize, by reacting either with atmospheric oxygen or with dissolved oxygen in the aqueous solution, with consequent production of non-desirable degradation products.

Paracetamol is an active ingredient that has been widely used in a large number of pharmaceutical preparations over the last decades. It is commonly used as an analgesic and antipyretic. However, due to its lack of solubility in water and due to the fact that paracetamol in aqueous medium is unstable in the presence of oxygen and/or light, it is difficult to obtain a pharmaceutical ready-to-use solution for intravenous perfusion.

Moreover, some of the resultant oxidation products may be hepatotoxic in humans, such as benzoquinoimines, and may additionally lead to the formation of coloured compounds, thus making the aqueous solution unsuitable for therapeutic applications.

Paracetamol can be decomposed through a plurality of degradation pathways.

The hydrolysis of paracetamol results in p-aminophenol which easily oxidizes resulting in strong pink coloured quinine imines (Fairbrother J. E., Acetominophen, in analytical profiles of drug substances (1974), volume 3, pages 1-109).

Paracetamol pH-rate profile reveals a specific acid and specific base catalysis with maximum stability in the pH range of 5 to 7.(K. T. Koshy and J. L. Lach, J. Pharm. Sci., Philadelphia, 1975).

It is also common knowledge that by blocking the action of oxygen by either eliminating or neutralizing it, or by combining both processes, it is possible to improve paracetamol stability considerably. According to WO01/93830A1 several methods may be employed:

i) Oxygen elimination can be achieved by increasing the temperature of the aqueous solution.

ii) Oxygen elimination can be achieved by submitting the aqueous solution to a vacuum.

iii) Oxygen elimination can be achieved by bubbling an inert gas such as nitrogen or argon through the solution.

iv) Neutralization of oxygen dissolved in the aqueous solution, by addition of an antioxidant agent.

v) Combining the elimination of oxygen with the addition of an antioxidant.

The patent application WO98/05314A1 describes paracetamol solutions in an aqueous solvent combined with a buffering agent having a pH of 4 to 8, and a free radical capturing agent. A water-insoluble inert gas is bubbled through the aqueous solvent to remove oxygen.

According to EP 858329 B1 the use of an antioxidant agent does not have a significant effect on paracetamol stability but it can prevent solution coloration,

The WO03/041687A2 application refers to a method for producing stabilized antioxidant-free solutions at low temperatures, which consists in deoxygenating the solutions by bubbling with an inert gas and in deoxygenating gas hold-ups of the vessels, of the manufacturing pipes and inerting of ampoules and flasks containing the solute with a dense inert gas.

Based on the precedent tests, deoxygenating the aqueous solvent by bubbling an inert gas contributes to the solution's stability, maintaining them almost uncoloured.

According to the above WO01/93830A1 application, the bubbling with an inert gas however only allows oxygen content to decrease to values of as low as 2 ppm maximum. Additionally, the aqueous formulations may contain an antioxidant agent and a hydroxypolycarboxylic acid or salt (for instance trisodium citrate or disodium tartrate). The presence of an antioxidant agent completes the deoxygenation effect but it does not substitute the deoxygenation. The addition of a hydroxypolycarboxylic acid decreases the antioxidant consumption and decreases the antioxidant concentration ranging from 0.1 mg to 1000 mg per one solution litre. The vial filling is performed under an atmosphere of an inert gas and the stoppering is performed under depression in order to obtain a pressure of less than the atmospheric pressure, until a maximum of 65000 Pa.

Surprisingly, the present invention now allows the preparation of stable pharmaceutical compositions containing paracetamol in an aqueous solvent, with an antioxidant, using a manufacturing process that does not require the use of bubbling devices, nor the increase of solution temperature to promote the solution deoxygenation. The manufacturing process does not require the use of low working temperatures to assure solution stability.

Instead the present invention leads to the manufacture of stable paracetamol solutions in an aqueous solution medium in a pH range of 4 to 8
1) where the deoxygenation process is achieved only by flushing the headspace of storage tanks with an inert gas and
2) with the use of an antioxidant to protect paracetamol from residual oxygen that might still be present in the solution, eliminating the need of low working temperatures,
   which is contrary to the prior art knowledge, and surprising as it was common understanding that only the deoxygenation by bubbling succeeded to produce stabilized solutions.

Furthermore, the present invention also leads to an energetically more efficient manufacturing process (no need to use high or low working temperatures), and eliminates the need for bubbling devices, decreasing the number of devices that are in direct contact with the solution, and also decreasing possible contamination problems. A deoxygenated paracetamol solution allows lower contents of antioxidant for paracetamol stabilization, rationalizing the use of antioxidants, and other stabilizing excipients present in the pharmaceutical formulation.

### SUMMARY OF THE INVENTION

The present invention concerns a novel method for producing stabilized solutions based on paracetamol, consisting of protecting the solution against possible oxygen uptake by maintaining them under inert gas atmosphere during the manufacturing process and after the filling of the final containers, to obtain aqueous solutions having a residual oxygen concentration in the solution below 2 ppm, and preferably of the order of 1 ppm and even 0.5 ppm and an oxygen concentration in the final container headspace (gas phase) below 10%, and preferably in the order of 3% and even virtually oxygen free, after the filling process.

### DETAILED DESCRIPTION

In the following, the present invention will be explained in greater detail:

1—A liquid stable paracetamol formulation in an aqueous solvent, comprising at least one of the following excipients: an antioxidant, a polyol, one or more buffering agent(s), a stabilizer, a base or an acid for adjustment of the pH of said formulation to 4 to 8 and, wherein the formulation in said solvent is deoxygenated by flushing an inert gas into the headspace of the tank and into the headspace of the final container.

2—A liquid stable paracetamol formulation in an aqueous solvent according to item 1 wherein the antioxidant is selected from the group consisting of ascorbic acid, sodium acetate, sodium metabisulphite, organic compounds which bear at least one thiol function, citrates, preferentially sodium citrate and citric acid, cysteine and/or acetylcysteine.

3—A liquid stable paracetamol formulation in an aqueous solvent according to any one of the items 1 to 2 wherein the antioxidant is sodium metabisulphite.

4—A liquid stable paracetamol formulation in an aqueous solvent according to any one of the items 1 to 2 wherein the antioxidant is sodium citrate.

5—A liquid stable paracetamol formulation in an aqueous solvent according to any one of the items 1 to 2 wherein the antioxidant is cysteine.

6—A liquid stable paracetamol formulation in an aqueous solvent according to any one of items 1 to 5 wherein the polyol is a polyhydroxylated alcohol or a sugar alcohol.

7—A liquid stable paracetamol formulation in an aqueous solvent according to item 6 wherein the polyol is mannitol.

8—A liquid stable paracetamol formulation in an aqueous solvent according to any one of items 1 to 7 wherein the stabilizer is magnesium chloride.

9—A liquid stable paracetamol formulation in an aqueous solvent, wherein the formulation in said solvent is deoxygenated by decreasing the partial pressure of oxygen present in the headspace of a tank and the final container by flushing nitrogen, in which the pH is adjusted to 4 to 8 by the addition of a buffering agent and wherein the formulation comprises at least one of the following excipients: water, cysteine, sodium citrate, sodium phosphate, magnesium chloride, mannitol, and, a base or an acid for adjustment of the pH of said formulation to 4 to 8.

10—A liquid stable paracetamol formulation in an aqueous solvent according to claim 9 wherein the solution further comprises sodium metabisulphite.

11—A liquid stable paracetamol formulation in an aqueous solvent, wherein the formulation in said solvent is deoxygenated by decreasing the partial pressure of oxygen present in the headspace of a tank and the final container by flushing argon, in which the pH of the aqueous solution is adjusted to 4 to 8 by the addition of a buffering agent and wherein the formulation comprises at least one of the following excipients: water, cystein, sodium citrate, sodium phosphate, magnesium chloride, mannitol and, a base or an acid for adjustment of the pH to 4 to 8.

12—A liquid stable paracetamol formulation in an aqueous solvent according to item 11 wherein the solution further comprises sodium metabisulphite.

13—A liquid stable paracetamol formulation in an aqueous solvent according to any one of the items 1 to 8 wherein the buffering agent is selected from the group consisting of phosphates and/or citrates.

14—A liquid stable paracetamol formulation in an aqueous solvent according to item 13 wherein the buffering agent is sodium phosphate.

15—A liquid stable paracetamol formulation in an aqueous solvent according to item 13 wherein the buffering agent is sodium citrate.

16—A liquid stable paracetamol formulation in an aqueous solvent according to any one of the items 1 to 15 wherein the concentration of paracetamol is not less than 5 mg/ml.

17—A liquid stable paracetamol formulation in an aqueous solvent according to any one of the items 1 to 15 wherein the concentration of paracetamol is not more than 15 mg/ml.

18—A process for producing a liquid stable paracetamol formulation in an aqueous solvent, wherein the formulation in said solvent] is deoxygenated by flushing an inert gas into the headspace of a tank and into the headspace of the final container and wherein said formulation comprises at least one of the following excipients: water, an antioxidant, a polyol, one or more buffering agent, a stabilizer, a base or an acid for final adjustment of the pH.

19—A process for producing a liquid stable paracetamol formulation in an aqueous solvent according to item 18 wherein the inert gas is argon, nitrogen, helium or neon.

20—A process for producing a liquid stable paracetamol formulation in an aqueous solvent according to items 18 and 19 wherein the inert gas is argon.

21—A process for producing a liquid stable paracetamol formulation in an aqueous solvent according to items 19 and 20 wherein the inert gas is nitrogen.

22—A process for producing a liquid stable paracetamol formulation in an aqueous solvent, wherein the formulation in said solvent is deoxygenated by decreasing the partial pressure of oxygen present in the headspace of the tank and in the headspace of the final container by flushing nitrogen, in which the pH of the formulation in said solvent is adjusted to 4 to 8 by the addition of a buffering agent and the formulation comprises at least one of the following excipients: water, cysteine, sodium citrate, sodium phosphate, magnesium chloride, mannitol, and, a base or an acid for adjustment of the pH to 4 to 8.

23—A process for producing a liquid stable paracetamol formulation in an aqueous solvent according to item 22 wherein the solution further comprises sodium metabisulphite.

24—A process for producing a liquid stable paracetamol formulation in an aqueous solvent, wherein the formulation in said solvent is deoxygenated by decreasing the partial pressure of oxygen present in the headspace of the thank and in the headspace of the final container by flushing argon, in which the pH of the formulation is adjusted to 4 to 8 by the addition of a buffering agent and the aqueous solution comprises at least one of the following excipients: water, cysteine, sodium citrate, sodium phosphate, magnesium chloride, mannitol, and, a base or an acid for adjustment of the pH to 4 to 8.

25—A process for producing a liquid stable paracetamol formulation in an aqueous solvent according to item 24 wherein the solution further comprises sodium metabisulphite.

26—A process for producing a liquid stable paracetamol formulation in an aqueous solvent according to items 18 to 25 wherein the concentration of paracetamol is not less than 5 mg/ml.

27—A process for producing a liquid stable paracetamol formulation in an aqueous solvent according to items 18 to 25 wherein the concentration of paracetamol is not more than 15 mg/ml.

28—Use of a liquid stable paracetamol formulation in an aqueous solvent according to any one of the items 1 to 17 for the treatment of pain and fever.

29—Use of a liquid stable paracetamol formulation in an aqueous solvent obtained by any one of the items 18 to 27 for the treatment of pain and fever.

It is explicitly pointed out that although the present invention is described in the above with reference to separate items, all of these describe preferred aspects which can be combined with each other to further improve the achievements of the present invention.

According to the present invention, the aqueous solvent is preferably water. It is known that paracetamol is very slightly soluble in water (“The Merck Index”, 12th edition, page 9, no 45, 1996).

The liquid pharmaceutical compositions according to the present invention are preferentially injectable compositions. The paracetamol concentration in solution is preferably comprised between 2 mg/ml and 50 mg/ml.

The present inventive manufacturing process does not involve bubbling the aqueous solution or solvent with an inert gas. The compositions according to the invention are prepared with water for injections with a dissolved oxygen content of lower then 8.8 mg/l.

According to the invention all manufacturing storage tanks and pipes used during the manufacturing process are cleared of any oxygen contained therein, for example by the insufflation of an inert gas.

### Definitions

An antioxidant in the present context is a molecule capable of slowing or preventing the oxidation of other molecules. The aqueous solution stability is influenced by the presence of an antioxidant such as cysteine, acetylcysteine, dithiothreitol, thiomalic acid, thioglycerol or methionine, sodium metabisulphite, ascorbic acid, sodium acetate, citric acid or sodium citrate. The antioxidant completes the deoxygenation of the solution. Without an antioxidant, an essentially deoxygenated solution becomes pink in colour after a certain time at ambient temperature, for the reasons as explained above.

A thiol in the present context is a compound that contains a functional group composed of a sulphur-hydrogen (—SH). Being the sulphur analogue of an alcohol group (—OH), this functional group is referred to either as a thiol group or a sulphydryl group. The compounds cysteine and glutathione are examples of thiols.

A stabilizer in the present context is a chemical substance which tends to inhibit the reaction between two or more other chemicals. Surprisingly, an increase in solution stability occurs, when a stabilizer, like e.g. magnesium chloride is added to the present paracetamol solution. Magnesium is also available in other forms such as oxide, gluconate, malate, citrate, sulphate, etc.

A buffering agent adjusts the pH of the solution. These agents are added to substances that are to be placed into acidic or basic conditions in order to stabilize the substance. The presently employed buffer is compatible with human injectable administration, in order to establish the pH between 4 and 8, or preferably 5 to 7, even more preferably close to 6, the optimal pH for paracetamol aqueous solution stability. An appropriate buffer will be, for example, a sodium hydrogenphosphate, disodic phosphate, sodium acetate, sodium citrate or trisodic citrate buffer. The buffer concentration may be comprised between 0.1 and 10 mg/ml.

A base in the present context is any chemical compound that, when dissolved in water, results in a solution with a hydrogen ion activity which is lower than that of pure water. The employed base is compatible with human injectable administration, in order to establish the pH between 4 and 8, preferably 5 to 7, even more preferably close to 6, the optimal pH for paracetamol aqueous solution stability. An appropriate base will be for example, sodium hydroxide.

An acid in the present context is any chemical compound that, when dissolved in water, results in a solution with a hydrogen ion activity which is higher than that of pure water. The employed acid is compatible with human injectable administration, in order to establish the pH between 4 and 8, preferably 5 to 7, even more preferably close to 6, the optimal pH for paracetamol aqueous solution stability. An appropriate acid will be for example, hydrochloric acid.

A polyol in the present context is a compound containing more than one hydroxyl group (OH). Each hydroxyl group is attached to an aliphatic skeleton. The presently employed polyol is compatible with human injectable administration. Mannitol, sorbitol, and/or xylitol are examples of polyols.

According to the present invention the tanks used in the manufacturing process, are preferably standard stainless steel tanks, typically used by the current art of manufacturing pharmaceutical compositions. The head-space of the tank is the volume left at the top of an almost filled tank. All references to ‘tanks’ in the present application are meant to refer to the above-described tanks as used during manufacture of a paracetamol formulation.

According to the present invention the final containers used in the manufacturing process, are standard containers of glass or plastic, typically used by the current art of manufacturing pharmaceutical compositions. The head space of the final container is the volume left at the top of an almost filled final container such as ampoules, injection bottles, infusion bottles, etc. Thus all references to ‘containers’ in the present application refer to those containers as defined above, used during manufacture and/or (final) storage of the paracetamol formulation.

Inert gas is a non-reactive gas used during preservation of reactive materials. Nitrogen and argon are the most common inert gases for use in chemistry. According to the present invention a stable liquid paracetamol formulation can be achieved using an inert gas or a mixture of inert gases in the manufacturing process.

According to the present invention, manufacturing pipes used in the present invention are standard pipes typically used by the current art of manufacturing pharmaceutical compositions.

Within the framework of the industrial manufacture of injectable solutions, it is common to use heat sterilization. In spite of precautions that may be taken to deoxygenate the injectable solution at an initial point of manufacture, the solution can once again take up significant quantities of dissolved oxygen during the subsequent manufacturing steps. If these solutions are subjected to heat-sterilization, especially at high temperatures in the region of 120° C., the residual quantity of dissolved oxygen can easily react with paracetamol, resulting in its total or partial degradation. According to the present invention the preparation is subjected to sterilization by filtration under inert gas before this solution is introduced into the container. The solution is introduced into the container under aseptic conditions, under a sterile inert gas. The sterilization by filtration allows overcoming the problem of paracetamol oxidation during the sterilization process.

According to the present invention, before the paracetamol aqueous solution is introduced into the final container, the latter is cleared of the air contained therein, for example by the insufflation of an inert gas.

Once the containers have been filled, they are stoppered under an inert gas atmosphere, preferably nitrogen at a pressure of preferably 1 atm.

According to the present invention, the deoxygenation process used is performed without any bubbling of the aqueous solution with an inert gas. Regardless of this fact, the deoxygenation process is still efficient and adequate in order to assure sufficient deoxygenation of the paracetamol aqueous solution.

According to the present invention, the paracetamol aqueous solution's stability is significantly increased, by maintaining the solution under an inert gas atmosphere during the manufacturing process and after the filling of the final containers.

In a preferred embodiment, the headspace of the tank(s) and of the container(s) is almost completely deoxygenated. In an even more preferred embodiment, the headspace of the tank(s) and the container(s) is completely deoxygenated, i.e. all possible headspace involved in manufacture and storage are deoxygenated.

Preferably, the final liquid paracetamol formulation will be isotonized.

### EXAMPLES

To study the parameters that affect the stability of a liquid paracetamol solution, several test solutions have been made, regarding different formulations and different manufacturing processes.

In order to perform the paracetamol assay in test solutions, the following validated method was used:

Chromatographic system:

| | |
|---|---|
| Column | betabasic C8 250 × 4.6 mm, 5 μm |
| Flow | 1.2 ml/min |
| Wavelength | 245 nm |
| Temperature | 35° C. |
| Injection volume | 40 μl |
| Run time | 15 minutes |
| Mobile phase | 375 Volumes of solution A: 375 Volumes of |
| | Solution B: 250 Volumes of Solution C |

Mobile phase:

Solution A: Weigh 17.9 g of disodium hydrogen phosphate (Na₂HPO₄) and dilute in 1000 mL of water.

Solution B: Weigh 7.8 g of sodium di-hydrogen phosphate monohydrate (NaH₂PO4.H₂O) and dilute in 1000 mL of water.

Solution C: Dissolve 4g of tetrabutylammonium hydroxide in 10 mL of water. Sonicate for 5 minutes. Weigh 4.6 g of this solution and dilute in 1000 mL of methanol.

Retention time of Paracetamol in this chromatographic system: 4.00 minutes Note: The flow should be adjusted in order to obtain a paracetamol retention time of 4 minutes.

Standard solution: Dissolve 25 mg of a paracetamol chemical reference substance (European Pharmacopeia) to a 50 mL volumetric flask and complete the volume with mobile phase (500 μg/mL). Sonicate for 5 minutes. Transfer 3 mL of this solution to a 50 mL volumetric flask and complete the volume with mobile phase.

C_{paracetamol}=30 μg/mL.

Sample solution: Take 5 mL of a paracetamol test solution (See Table 1)[3] to a 10 mL volumetric flask and complete the volume with mobile phase. Pipette 0.300 mL of this solution to a 50 mL volumetric flask and complete the volume with mobile phase.

The paracetamol assay is expressed in percentage of paracetamol theoretical concentration in the test solution to be analyzed.

In order to determine paracetamol-related substances, i.e. impurities present in the test solutions, the following validated method was used:

Chromatographic system:

| | |
|---|---|
| Column | betabasic C8 250 × 4.6 mm, 5 μm |
| Flow | 1.2 ml/min |
| Wavelength | 245 nm |
| Temperature | 35° C. |
| Injection volume | 40 μl |
| Run time | 50 minutes (12 times the retention time of |
| | paracetamol) |
| Mobile phase | 375 Volumes of solution A: 375 Volumes of |
| | Solution B: 250 Volumes of Solution C |

Mobile phase:

Solution A: Weigh 17.9 g of disodium hydrogen phosphate (Na₂HPO₄) and dilute in 1000 mL of water.

Solution B: Weigh 7.8 g of sodium di-hydrogen phosphate monohydrate (NaH₂PO₄.H₂O) and dilute in 1000 mL of water.

Solution C: Dissolve 4 g of tetrabutylammonium hydroxide in 10 mL of water. Sonicate for 5 minutes. Weight 4.6 g of this solution and dilute in 1000 mL of methanol.

System suitability Solution: Dissolve 5.0 mg of 4-aminophenol, 5 mg of standard paracetamol and 5.0 mg of chloroacetanilide in methanol and dilute in a 20 ml volumetric flask with the same solvent. Dilute 0.5 ml in a 250.0 ml volumetric flask with mobile phase.

C_{4-Aminophenol}=0.5 μg/mL; C_{paracetamol}=0.5 μg/mL; C_{chloroacetanilide}=0.5 μg/mL.

Standard solution: Dissolve 25 mg of paracetamol chemical reference substance (European Pharmacopeia) to a 50 mL volumetric flask and complete the volume with mobile phase (500 μg/mL). Sonicate for 5 minutes. Transfer 1 mL of this solution to a 100 mL volumetric flask and complete the volume with mobile phase.

C_{paracetamol}=5 μg/mL

Sample solution: Transfer 5 mL of paracetamol test solution (See Table 1) to a 10 mL volumetric flask and complete the volume with mobile phase.

Paracetamol-related substances are expressed in percentage of paracetamol theoretical concentration in the test solution to be analyzed.

The European Pharmacopoeia monograph 2.2.2. Degree of coloration of Liquids test was performed in order to determine the paracetamol solution degree of coloration, using the yellow reference solutions from Y7 to Y1. The reference Y7 being the least intense degree of coloration and the Y1 being the most intense degree of coloration.

The test solutions were submitted to photostability tests in an Atlas Suntest device working at 500 W/m².

The test solution's composition is described in Table 1

TABLE 1
Test solution composition.

| | Test solution | | | | | | |
|---|---|---|---|---|---|---|---|
| Components | A | B | C | D | E | F | G |
| Paracetamol | Yes | Yes | Yes | Yes | Yes | Yes | Yes |
| Water | Yes | Yes | Yes | Yes | Yes | Yes | Yes |
| Sodium hydroxide/ | Yes | Yes | Yes | Yes | Yes | Yes | Yes |
| Hydrochloric acid | | | | | | | |
| Sodium | Yes | Yes | Yes | Yes | Yes | Yes | Yes |
| Hydrogenphosphate | | | | | | | |
| Mannitol | Yes | Yes | Yes | Yes | Yes | Yes | Yes |
| Cysteine | Yes | No | Yes | Yes | Yes | Yes | Yes |
| Sodium | No | No | No | Yes | No | No | No |
| metabisulphite | | | | | | | |
| Sodium citrate | No | No | No | No | Yes | No | No |
| Magnesium chloride | No | No | No | No | No | Yes | No |

Deoxygenation conditions:

a) Deoxygenated tank and bottle head-space: in the manufacturing process the deoxygenation of the head-space was performed in both tank and final container.

b) Partially deoxygenated: the manufacturing process here only contemplated the deoxygenation of the tank head-spaces. The final container (bottle) head-space was not deoxygenated.

### Example 1

### Effect of Cysteine in Paracetamol Solution Stabilization

It has been verified that the presence of cysteine has a stabilizing effect on paracetamol, decreasing its degradation rate in aqueous solutions.

In this study the stability of 1% m/v paracetamol deoxygenated solutions was compared with (Solution A) and without (Solution B) cysteine. The solutions were additionally deoxygenated by flushing nitrogen into the head-space of the tank. The pH of the paracetamol solution was adjusted to 5.8. The paracetamol solution was filled in glass bottles under nitrogen atmosphere. The stability was evaluated under photostability stress conditions over 48 hours at 40° C.

The stability was evaluated in terms of percentage of impurities formed in the solution and colour of the solution. The colour of the solution is related to the presence of p-aminophenol, benzoquinoneimines, polymerization products and other impurities related to the synthesis and degradation of paracetamol.

The results are described in Table 2.

TABLE 2
Results for solutions A and B

| Test | Solution A | Solution B |
|---|---|---|
| Antioxidant | Cysteine | None |
| Deoxygenated tank | Yes | Yes |
| and bottle head- | | |
| space | | |
| | After aseptic filling | |
| Colour | <Y7 [19] (colourless) | <Y7 (colourless) |
| EP yellow (Y) | | |
| reference | | |
| | After photostability test for 48 hours/40° C. | |
| Colour | <Y7 (colourless) | <Y7 (colourless) |
| % Impurity, individual | 0.006 | 0.117 |
| maximum value | | |
| % Total impurities | 0.006 | 0.134 |

It can be observed that cysteine used as antioxidant improves paracetamol stability, preventing the formation of impurities, i.e. degradation products of paracetamol.

### Example 2

### The Effect of Dissolved Oxygen on Paracetamol Solution Stability

It has been verified that paracetamol aqueous solutions are unstable in the presence of oxygen. Therefore, decreasing the dissolved oxygen content in paracetamol solution prevents the degradation of paracetamol.

In this study, the stability of 1% m/v paracetamol deoxygenated solutions (solution A) against 1% m/v paracetamol solutions with an uncompleted extent of deoxygenation i.e. partial deoxygenated solution (solution C) was compared.

To that end solution A was deoxygenated by flushing nitrogen into the head-space of the tank. The pH of paracetamol solution was adjusted to 5.8. The paracetamol solution was filled in glass bottles under nitrogen atmosphere.

Solution C was prepared and the pH of the paracetamol solution was adjusted to 5.8. The paracetamol solution was filled in glass bottles.

The stability was evaluated under photostability stress conditions over 48 hours at 40° C.

The colour of solution is related to the presence of p-aminophenol, benzoquinoneimines, polymerization products and other impurities related to the synthesis and degradation of paracetamol.

The results are shown in Table 3

TABLE 3
Results for solutions A and C

| Test | Solution A | Solution C |
|---|---|---|
| Antioxidant | Cysteine | Cystein |
| Deoxygenated tank | Yes | Partial deoxygenated |
| and bottle head- | | |
| space | | |
| | After aseptic filling | |
| Colour | <Y7 (colourless) | <Y7 (colourless) |
| EP yellow (Y) | | |
| reference | | |
| | After photostability test for 48 hours/40° C. | |
| Colour | <Y7 (colourless) | <Y3 (intense yellow) |

It can be observed that a decrease in oxygen content in the final container leads to an increase in paracetamol solution stability, preventing colour formation under stress stability conditions.

### Example 3

### Effect of Sodium Metabisulphite in Paracetamol Solution Stabilization

It could be shown by the inventors that the presence of sodium metabisulphite has a stabilizing effect on paracetamol, decreasing its degradation rate in aqueous solutions.

In this study, the stability of 1% m/v paracetamol non-deoxygenated solutions with (solution D) and without sodium metabisulphite (solution C) was compared. The pH of paracetamol solution was adjusted to 5.8. The paracetamol solution was filled in glass bottles. The stability was evaluated under photostability stress conditions over 48 hours at 40° C.

The stability was evaluated in terms of colour of solution. The colour of solution is related to the presence of p-aminophenol, benzoquinoneimines, polymerization products and other impurities related to the synthesis and degradation of paracetamol.

The results are shown in Table 4.

TABLE 4
Results for solutions D and C

| Test | Solution D | Solution C |
|---|---|---|
| Sodium | Yes | No |
| metabisulphite | | |
| Deoxygenated tank | Partial deoxygenated | Partial deoxygenated |
| and bottle head- | | |
| space | | |
| | After aseptic filling | |
| Colour | <Y7 (colourless) | <Y7 (colourless) |
| EP yellow (Y) | | |
| reference | | |
| | After photostability test for 48 hours/40° C. | |
| Colour | <Y7 (colourless) | <Y3 (intense yellow) |

It can be observed that sodium metabisulphite used as antioxidant improves paracetamol stability.

### Example 4

### Effect of Sodium Citrate in Paracetamol Solution Stabilization

It could be shown by the present inventors that the presence of sodium citrate has a stabilizing effect on paracetamol, decreasing its degradation rate in aqueous solutions.

In this study, the stability of 1% m/v paracetamol non-deoxygenated solutions with (Solution E) and without (Solution C) sodium citrate was compared. The pH of paracetamol solution was adjusted to 5.8. The paracetamol solution was filled in glass bottles under a partially deoxygenated atmosphere. The stability was evaluated under photostability stress conditions over 48 hours at 40° C.

The stability was evaluated in terms of colour of solution. The colour of solution is related to the presence of p-aminophenol, benzoquinoneimines, polymerization products and other impurities related to the synthesis and degradation of paracetamol.

The results are shown in Table 5.

TABLE 5
Results for solutions E and C

| Test | Solution E | Solution C |
|---|---|---|
| Sodium citrate | Yes | No |
| Deoxygenated tank | Partial deoxygenated | Partial deoxygenated |
| and bottle head- | | |
| space | | |
| | After aseptic filling | |
| Colour | <Y7 (colourless) | <Y7 (colourless) |
| EP yellow (Y) | | |
| reference | | |
| | After photostability test for 48 hours/40° C. | |
| Colour | <Y4 (yellow) | <Y3 (intense yellow) |

It can be observed that sodium citrate used as antioxidant improves paracetamol stability.

### Example 5

### Effect of Magnesium Chloride in Paracetamol Solution Stabilization

It could be shown by the present inventors that the presence of magnesium chloride has a stabilizing effect on paracetamol, decreasing its degradation rate in aqueous solutions.

In this study, the stability of 1% m/v paracetamol non-deoxygenated solutions with (Solution F) and without (Solution C) magnesium chloride was compared. The pH of paracetamol solution was adjusted to 5.8. The paracetamol solution was filled in glass bottles under a partially deoxygenated atmosphere. The stability was evaluated under photostability stress conditions over 48 hours at 40° C.

The stability was evaluated in terms of percentage of colour of solution. The colour of solution is related to the presence of p-aminophenol, benzoquinoneimines, polymerization products and other impurities related to the synthesis and degradation of paracetamol.

The results are shown in Table 6.

TABLE 6
Results for solutions F and C

| Test | Solution F | Solution C |
|---|---|---|
| Magnesium chloride | Yes | No |
| Deoxygenated tank | Partial deoxygenated | Partial deoxygenated |
| and bottle head- | | |
| space | | |
| | After aseptic filling | |
| Colour | <Y7 (colourless) | <Y7 (colourless) |
| EP yellow (Y) | | |
| reference | | |
| | After photostability test for 48 hours/40° C. | |
| Colour | <Y4 (yellow) | <Y3 (intense yellow) |

It can be observed that magnesium chloride used as antioxidant improves paracetamol stability.

### Example 6

### Stability of Paracetamol Solution Over 10 Months at 25° C./60% RH

In this study, the stability of a 1% m/v paracetamol deoxygenated solution (Solution G) was studied. The pH of the paracetamol solution was adjusted to 5.8. The paracetamol solution was deoxygenated by flushing an inert gas into the headspace of the tank and into the headspace of the final container. The stability was evaluated over 10 months at 25° C./60% RH.

Stability results after storage at 25° C./60% RH for 10 months:

Paracetamol assay: 100.29% [

Impurity chloroacetanilide: not detected

Impurity 4-aminophenol: 0.004%

Impurity 4-nitrophenol: 0.005%

Largest single unknown impurity: 0,037%

Total impurities: 0.054%

Appearance: colourless (<Y7) clear solution

This is an excellent result compared to usual stability data for liquid paracetamol formulations.

1- A liquid stable paracetamol formulation in an aqueous solvent, comprising at least one of the following excipients: an antioxidant, a polyol, one or more buffering agent(s), a stabilizer, a base or an acid for adjustment of the pH of said formulation to 4 to 8 and, wherein the formulation in said solvent is deoxygenated by flushing an inert gas into the headspace of the tank and into the headspace of the final container.

2- A liquid stable paracetamol formulation in an aqueous solvent according to claim 1 wherein the antioxidant is selected from the group consisting of ascorbic acid, sodium acetate, sodium metabisulphite, organic compounds which bear at least one thiol function, citrates, preferentially sodium citrate and citric acid, cysteine and/or acetylcysteine.

3- A liquid stable paracetamol formulation in an aqueous solvent according to any one of the claims 1 to 2 wherein the antioxidant is sodium metabisulphite.

4- A liquid stable paracetamol formulation in an aqueous solvent according to any one of the claims 1 to 2 wherein the antioxidant is sodium citrate.

5- A liquid stable paracetamol formulation in an aqueous solvent according to any one of the claims 1 to 2 wherein the antioxidant is cysteine.

6- A liquid stable paracetamol formulation in an aqueous solvent according to any one of claims 1 to 5 wherein the polyol is a polyhydroxylated alcohol or a sugar alcohol.

7- A liquid stable paracetamol formulation in an aqueous solvent according to claim 6 wherein the polyol is mannitol,

8- A liquid stable paracetamol formulation in an aqueous solvent according to any one of claims 1 to 7 wherein the stabilizer is magnesium chloride.

9- A liquid stable paracetamol formulation in an aqueous solvent, wherein the formulation in said solvent is deoxygenated by decreasing the partial pressure of oxygen present in the headspace of a tank and the final container by flushing nitrogen, in which the pH is adjusted to 4 to 8 by the addition of a buffering agent and wherein the formulation comprises at least one of the following excipients: water, cysteine, sodium citrate, sodium phosphate, magnesium chloride, mannitol, and, a base or an acid for adjustment of the pH of said formulation to 4 to 8.

10- A liquid stable paracetamol formulation in an aqueous solvent according to claim 9 wherein the solution further comprises sodium metabisulphite.

11- A liquid stable paracetamol formulation in an aqueous solvent, wherein the formulation in said solvent is deoxygenated by decreasing the partial pressure of oxygen present in the headspace of a tank and the final container by flushing argon, in which the pH of the aqueous solution is adjusted to 4 to 8 by the addition of a buffering agent and wherein the formulation comprises at least one of the following excipients: water, cystein, sodium citrate, sodium phosphate, magnesium chloride, mannitol and, a base or an acid for adjustment of the pH to 4 to 8.

12- A liquid stable paracetamol formulation in an aqueous solvent according to claim 11 wherein the solution further comprises sodium metabisulphite.

13- A liquid stable paracetamol formulation in an aqueous solvent according to any one of the claims 1 to 8 wherein the buffering agent is selected from the group consisting of phosphates and/or citrates.

14- A liquid stable paracetamol formulation in an aqueous solvent according to claim 13 wherein the buffering agent is sodium phosphate.

15- A liquid stable paracetamol formulation in an aqueous solvent according to claim 13 wherein the buffering agent is sodium citrate.

16- A liquid stable paracetamol formulation in an aqueous solvent according to any one of the claims 1 to 15 wherein the concentration of paracetamol is not less than 5 mg/ml.

17- A liquid stable paracetamol formulation in an aqueous solvent according to any one of the claims 1 to 15 wherein the concentration of paracetamol is not more than 15 mg/ml.

18- A process for producing a liquid stable paracetamol formulation in an aqueous solvent, wherein the formulation in said solvent is deoxygenated by flushing an inert gas into the headspace of a tank and into the headspace of the final container and wherein said formulation comprises at least one of the following excipients: water, an antioxidant, a polyol, one or more buffering agent, a stabilizer, a base or an acid for final adjustment of the pH.

19- A process for producing a liquid stable paracetamol formulation in an aqueous solvent according to claim 18 wherein the inert gas is argon, nitrogen, helium or neon.

20- A process for producing a liquid stable paracetamol formulation in an aqueous solvent according to claims 18 and 19 wherein the inert gas is argon.

21- A process for producing a liquid stable paracetamol formulation in an aqueous solvent according to claims 19 and 20 wherein the inert gas is nitrogen.

22- A process for producing a liquid stable paracetamol formulation in an aqueous solvent, wherein the formulation in said solvent is deoxygenated by decreasing the partial pressure of oxygen present in the headspace of the tank and in the headspace of the final container by flushing nitrogen, in which the pH of the formulation in said solvent is adjusted to 4 to 8 by the addition of a buffering agent and the formulation comprises at least one of the following excipients: water, cystein, sodium citrate, sodium phosphate, magnesium chloride, mannitol, and, a base or an acid for adjustment of the pH to 4 to 8.

23- A process for producing a liquid stable paracetamol formulation in an aqueous solvent according to claim 22 wherein the solution further comprises sodium metabisulphite.

24- A process for producing a liquid stable paracetamol formulation in an aqueous solvent, wherein the formulation in said solvent is deoxygenated by decreasing the partial pressure of oxygen present in the headspace of the tank and in the headspace of the final container by flushing argon, in which the pH of the formulation is adjusted to 4 to 8 by the addition of a buffering agent and the aqueous solution comprises at least one of the following excipients: water, cysteine, sodium citrate, sodium phosphate, magnesium chloride, mannitol, and, a base or an acid for adjustment of the pH to 4 to 8.

25- A process for producing a liquid stable paracetamol formulation in an aqueous solvent according to claim 24 wherein the solution further comprises sodium metabisulphite.

26- A process for producing a liquid stable paracetamol formulation in an aqueous solvent according to claims 18 to 25 wherein the concentration of paracetamol is not less than 5 mg/ml.

27- A process for producing a liquid stable paracetamol formulation in an aqueous solvent according to claims 18 to 25 wherein the concentration of paracetamol is not more than 15 mg/ml.

28- Use of a liquid stable paracetamol formulation in an aqueous solvent according to any one of the claims 1 to 17 for the treatment of pain and fever.

29- Use of a liquid stable paracetamol formulation in an aqueous solvent obtained by any one of the claims 18 to 27 for the treatment of pain and fever.
